(19) United States
(12) Patent Application Publication
(10) Pub. No.: US 2011/0244033 A1
(43) Pub. Date: Oct. 6, 2011

(54) TAMSULOSIN PELLETS FOR FIXED DOSE COMBINATION

(76) Inventors: Denny Johan Marijn Van Den Heuvel, Beuningen (NL); Johannes WilhelmusMaurice Weijers, Wijchen (NL); Dirk Pamperin, Nijmegen (NL)

(21) Appl. No.: 13/133,877

(22) PCT Filed: Dec. 9, 2008
(86) PCT No.: PCT/EP2008/010446

§ 371 Date: Jun. 22, 2011

(51) Int. Cl. A61K 9/48; A61K 31/18; A61K 31/473; A61P 13/08; A61P 9/00

(52) U.S. Cl. 424/451; 514/603; 514/284

(57) ABSTRACT

The invention relates to a population of tamsulosin-comprising pellets for oral administration of a combination dosage form containing physically separated a tamsulosin dose in the form of the population of the tamsulosin comprising pellets and at least one other dose of a pharmaceutically active substance, said pellets comprising tamsulosin hydrochloride uniformly dispersed in a carrier matrix, wherein (i) said pellets in the population have a size of less than about 1.4 mm and, advantageously, at least 90% of the pellets have a size of larger than 0.30 mm; and (ii) an average content of tamsulosin hydrochloride in the population of pellets is between about 0.15-3.00 weight percent, calculated on a dry pellet basis, to a process of making such population of pellets, and to their use.

### BACKGROUND OF THE INVENTION

The present invention relates to coated tamsulosin pellets and to unit dosage forms made therefrom.

Tamsulosin is the common name for (R)-5-[2-[[2-(2-ethoxyphenoxy)ethyl]-amino]propyl]-2-methoxy-benzenesulfonamide of the formula (I).

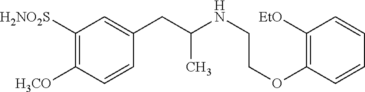

It is disclosed in EP 34432 and U.S. Pat. No. 4,731,478 as a pharmaceutically active substance having alpha-adrenergic blocking activity that is useful for treatment of cardiac insufficiencies and benign prostatic hyperplasia.

Tamsulosin hydrochloride medicaments are marketed under various tradenames, including FLOMAX® (Boehringer Ingelheim) in the U.S., HARNAL® (Yamanouchi) in Japan and OMNIC® (Yamanouchi) in Europe, for treatment of symptoms of benign prostatic hyperplasia (also known as BPH) such as urinary volume and frequency problems. The approved drug products include a capsule dosage form for oral administration that comprises 0.4 mg of the tamsulosin hydrochloride within a plurality of pellets. The capsule provides controlled release of the tamsulosin from the pellets and is a once daily dosage form, although two capsules can be used if needed; i.e. a maximum single daily administration of 0.8 mg. U.S. Pat. No. 4,772,475 is listed in the U.S. Food and Drug Administration's Approved Drug Products with Therapeutic Equivalence Evaluations (the “Orange Book”) as corresponding to FLOMAX®.

U.S. Pat. No. 4,772,475 (EP 194838, EP 533297) discloses controlled-release pharmaceutical dosage forms comprising multiple granulate units containing tamsulosin, microcrystalline cellulose and a release control agent. The granulate gradually releases tamsulosin from the granulate matrix. The patent suggests that an enteric coating is not needed.

WO 2004/043449 of Synthon discloses a pharmaceutical pellet composition comprising tamsulosin as an active ingredient and having an advantageous coating layer with respect to obtaining an extended release profile. Each pellet comprises a pellet core, which has a diameter within the range of 0.1-1.5 mm, comprising a tamsulosin salt, an inert pellet-forming carrier, a release control agent, and optionally water. Each pellet core is surrounded by an outer layer coat, which comprises a pharmaceutically acceptable acid-resistant polymer, in an amount, calculated on a dry pellet core basis, that is within the range of 1 to 25 mass %. The plurality of pellets exhibits a dissolution release profile in simulated gastric fluid using Ph. Eur. basket method at 100 rpm which includes releasing less than 25% of the tamsulosin during the first two hours. Preferably the pellet core contains from 0.05 to 5.0% of the tamsulosin salt (calculated in terms of tamsulosin hydrochloride). The mass of the outer layer coat is preferably within the range of 8-12%, calculated on a dry pellet core basis. All percentages are in mass %.

In a medical treatment, it is often considered as advantageous to administer tamsulosin together with another active substance. Such another substance might be of the same or different therapeutic class and may act in a synergistic way with tamsulosin. It is, of course, possible to administer two active substances separately but in some cases it is more advantageous to administer a fixed ratio of tamsulosin and another drug in a single dosage form. Many such suggestions were disclosed in the prior art.

According to the present medical experience, the therapeutical effect of tamsulosin is well pronounced when tamsulosin is administered in a polymeric matrix that modifies the release rate of tamsulosin in body fluids according to the therapeutical demands, whereby the release rate in the stomach is limited. Thus, the formulation of tamsulosin in a form of a coated monolithic pellets as suggested by WO 2004/043449 of Synthon, whereby the coating material prevents the release in the stomach and the matrix material modifies the release in the intestines, is advantageous from the therapeutical point of view and should be maintained also in a combination dosage form. On the other hand however, a second drug, which is to be co-administered, might require to exhibit a release rate that is not obtainable if such drug would be simply added to tamsulosin into the pellet matrix. In addition to, a second drug might react with tamsulosin or with the matrix material to yield undesirable side products and impurities. Third, a second drug might have properties, which would disallow to formulate it into pellets. In such a case, both drugs, although administered together and at the same time, should be administered in physically separated formulations within the final dosage form.

In an example, it is known that the active substance that might be co-administered with tamsulosin in a medical treatment, e.g. in a treatment of benign prostatic hyperplasia, is a testosterone-5α-reductase inhibitor, e.g. finasteride or dutasteride. WO 03090753 suggests the possibility of a combination medicament of tamsulosin and finasteride or dutasteride, however it provides no example of an actual combination composition that would actually be therapeutically effective and would have regard to different physical properties of both compounds. WO 2006055659 suggests a fixed dose composition (FDC) of dutasteride and tamsulosin, wherein dutasteride is formulated in a soft gel and tamsulosin is formulated in a form of beads, said beads comprising a multilayer composition with tamsulosin incorporated in one of these layers. Apparently, making a multilayer bead having reliable release rate of tamsulosing during its pathway in body fluids is technologically quite difficult and a simplification of the dosage form would be desirable.

A possible technical solution of this problem, which is however not a subject of the present application, is to make a dosage form, which would comprise an inner capsule loaded by the drug, which is to be co-administered in tamsulosin, and an outer capsule entirely covering the inner one, and to place the tamsulosin-containing coated pellets into the space between the inner and outer capsule. Then, after the administration, tamsulosin composition and the other medicament are independently liberated from the capsules (after sequentional dissolution of shells of both capsules) and then they interact with body fluids in their own therapeutically effective ways.

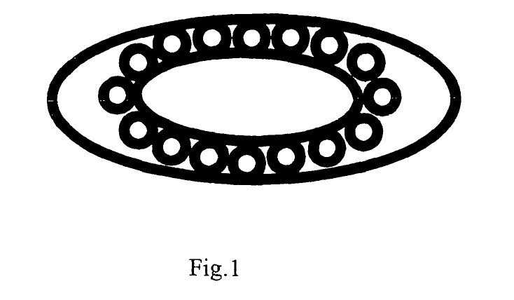

The technical solution is schematically shown on the FIG. 1

For to adapt tamsulosin pellets to be formulateable into such combination dosage form, several conditions must be fulfilled at the same time, particularly
the diameter of pellets is so adjusted to be able to fill effectively the space between the surfaces of both capsules
   the loading of tamsulosin in the respective pellets is so adjusted to obtain a population of certain, however quite limited, amount of pellets comprising, in total, the whole therapeutically effective dose of tamsulosin
   the qualitative and quantitative composition of the tamsulosin pellet matrix and/or coating is so adjusted to allow the desired release rate of tamsulosin after dissolution of the capsule.

A suitable technical solution of how to adapt the size and composition of tamsulosine pellets to pass the above requirements was not addressed in the prior art and thus it would be desirable to provide it.

### BRIEF DESCRIPTION OF THE INVENTION

The present invention provides a population of tamsulosin-comprising pellets adapted for oral administration of a combination dosage form containing physically separated a tamsulosin dose in the form of the population of the tamsulosin comprising pellets and at least one other dose of a pharmaceutically active substance, said pellets comprising tamsulosine hydrochloride uniformly dispersed in a carrier matrix, wherein

(i) said pellets in the population have a size of less than 1.4 mm and, advantageously, at least 90% of them have a size of larger than 0.30 mm and

(ii) an average content of tamsulosine hydrochloride in the population of pellets is between 0.15-3.00 weight percent, calculated on a dry pellet basis.

The invention also provides a process for making tamsulosin-comprising pellets of the above specification, comprising the steps of

a) pelletization of a mixture of 0.15-3.00% of tamsulosin hydrochloride with a matrix-forming material, followed by drying and coating the formed pellets cores by an acid resistant coat

b) sieving the population of pellets over a sieve of a pore diameter of 1.4 mm and collecting the population that passes through the sieve

c) optionally, sieving the population obtained in the step b) over a sieve of the pore diameter of 0.30 mm and collecting the population that do not pass through the sieve.

The invention also provides the use of tamsulosin pellets as defined above for making a medicament for co-administration of tamsulosin and at least one other pharmaceutically active substance in a fixed dose combination form.

### DETAILED DESCRIPTION OF THE INVENTION

It has been discovered that an effective modified release tamsulosin-comprising pellet population can be made that may be used for oral administration of a combination dosage form containing physically separated a tamsulosin dose in the form of the population of the tamsulosin comprising pellets and at least one other dose of a pharmaceutically active substance. In an example, the active substance that might be co-administered with tamsulosin, e.g. in a treatment of benign prostatic hyperplasia, is a testosterone-5α-reductase inhibitor. In particular, such dosage form may be advantageously represented by two concentrically placed capsules (“capsule-in-capsule”), wherein the inner, smaller one contains a pharmaceutical formulation comprising a dose of at least one active substance, which is to be co-administered with tamsulosin, and the space between the inner and outer capsule is filled with the population of tamsulosin pellets of the present invention, comprising, in total, the required dose of tamsulosin. Such dosage form shall be, in details, a subject of another patent application.

It has been found out that the suitable population of tamsulosin-comprising pellets, which is advantageously administrateable in a fixed-dose combination with another drug, is a population of pellets comprising tamsulosine hydrochloride uniformly dispersed in a carrier matrix, wherein:

(i) said pellets in the population have a size of less than 1.4 mm and, optionally, at least 90% of them have a size of more than 0.30 mm as well and

(ii) an average content of tamsulosine hydrochloride in the population of pellets is between 0.15-3.00 weight percent, calculated on the mass of the dried pellet

The pellets of the present invention include a pellet core which comprises tamsulosin hydrochloride and a carrier matrix comprising a pellet-forming carrier, a release control agent and, optionally, water.

The pellet-forming carrier is an inert material that is able to bind active ingredient and other excipients into an essentially spherical particulate material that is commonly called in pharmaceutical practice as a pellet. In the preferred composition of the pellet core, the microcrystalline cellulose (crystalline cellulose in other terminology) serves as a suitable inert carrier. Also alpha lactose, dextrin, mannitol, or chitosan, alone or in combination, may be used as pellet-forming carriers. Preferred amount of the pellet-forming carrier is 50-95 mass %, calculated on a dry pellet core basis.

The release control agent is an excipient which allows to release the active substance from the composition only under certain environmental conditions and/or by a certain release rate. Within the invention, the preferred agent is a pharmaceutically acceptable polymer, most preferably a water permeable polymer. For instance, various types of acrylic polymers, polyvinyl acetate and/or cellulose derivatives, (for instance ethyl cellulose, hydroxypropylmethylcellulose and modified analogues) may be used. Preferred amount of the release control agent/s in the composition is from 2.5 to 25 mass %, calculated on a dry pellet core basis.

An acrylic polymer is the preferred release control agent in the pellet core. Within the invention, an “acrylic polymer” means a pharmaceutically acceptable polymer of acrylic acid, such as sold under brand name Carbopol, or a copolymer of methacrylic acid and/or an acrylic or methacrylic acid ester, such as sold under brand name Eudragit. Such compounds are, e.g., defined in Handbook of Pharmaceutical excipients, edited by A.H.Kibbe, Pharmaceutical Press London, 3^{rd} ed. (2000). The release of the active substance from the admixture with such acrylic polymers may or may not be dependent on the environmental pH.

Preferably, the acrylic polymer is an acid-resistant acrylic polymer, which releases tamsulosin dependent upon the pH. Such polymers include Eudragit L products, especially Eudragit L 30 D. Eudragit L 30 D-55 is available as a 30% (m/V) aqueous dispersion of the acrylate polymer containing also polysorbate 80 and sodium lauryl sulphate as emulsifiers.

Alternatively, two types of release control agents may be combined together in order to induce both time-dependent and pH-dependent control of the release of tamsulosin. Use of agents that release the active substance independently of environmental pH prevents a dose dumping after the pellet core surface comes into contact with the body fluid, while agents releasing the active substance pH-dependently allow to focus the release of a main portion of the active component into desired part of gastrointestinal tract. An example of the polymer that releases substances independently of the pH is hydroxypropyl methylcellulose.

Making the pellet core is typically performed in the presence of a granulation liquid, which preferably comprises water. Water is the most suitable solvent and/or granulation liquid in the process of pellet formation, however it is almost completely removed afterwards. It is nevertheless important that water is preferably present in the dried composition of the core as it affects, sometimes significantly, the rate of diffusion once the coating has been dissolved in the intestinal fluid. Hence, the pellet core preferably requires water to remain in the dried cores, in an amount from to 2 to 10 mass %, and preferably from 2 to 5 mass %, calculated on a dry pellet core basis.

The “other” pharmaceutically acceptable excipients, if present, are generally used to provide proper characteristics of the composition within the pelletization procedure and include, inter alia, plasticizers (e.g. triethylcitrate) or an anti-sticking agent (e.g. talc).

The pellet core after drying typically comprises 0.2-2.5% mass of tamsulosin hydrochloride; 50-95% mass of microcrystalline cellulose; 1-25%, preferably 2.5-10%, mass of the acrylic polymer; 2-10%, preferably 2-5%, mass of water; and 0-25%, preferably 0.5-25% mass of other pharmaceutically acceptable excipients, calculated on the total mass of the dried core. As used herein the “dried core” means a core that has been substantially dried to be ready for coating and has a residual solvent content from the production thereof of 15% or less, more preferably 10% or less.

Additionally, the pellets of the present invention comprise a coating layer surrounding the pellet core, which comprises a pharmaceutically acceptable acid-resistant polymer material, preferably an acid-resistant acrylic polymer. Typically, the mass of said coating layer, calculated on a dry pellet core basis, is within the range of 2.5-17%, most preferably between 8-15% (w/w) of the weight of the dried pellet core.

The pharmaceutically acceptable acid-resistant material essentially protects the pellet core towards contact with gastric fluid and thus it minimizes the amount of tamsulosin that may be released in stomach. Preferred coating material comprises an acid resistant acrylic polymer. The “acid-resistant acrylic polymer” is a specific kind of the above acrylic polymer having free carboxyl groups. Such polymers are not soluble in acidic aqueous medium, while they are soluble in neutral or basic aqueous medium. Preferred acid resistant acrylic polymers include the Eudragit L series, such as Eudragit L 30 D-55. This acrylic polymer is available as an aqueous suspension, also comprising a small amount of emulsifiers, and may be directly used for coating in suitable coating equipment. In a particular aspect of the invention, the “acrylic polymer” used for the manufacturing of pellet core is advantageously identical with the “acid-resistant acrylic polymer” of the pellet coating.

The coating layer may, alternately or in combination, also comprise other acid resistant polymers such as cellulose acetate, cellulose acetate phthalate, hydroxypropylmethyl cellulose phthalate, hydroxypropylmethylcellulose acetate succinate etc. In addition, the coating composition may comprise other pharmaceutically acceptable excipients. For example, an anti-sticking agent, such as talc, may be added to the coating composition to avoid stickiness of the coated granules during the process. Similarly, a plasticizer such as triethylcitrate can improve the characteristics of the final film coat.

The amount of an acid resistant polymer, particularly the acrylic polymer, in the coating layer composition is preferably within the range of 25-95 mass %, more preferably 30 to 75%, and typically 50 to 75%, calculated on a dry basis of the coating layer. Preferably, the acrylic polymer is the only acid-resistant polymer in the outer layer coat. The remainder of the coating layer is pharmaceutically acceptable excipients and/or other acid-resistant polymer(s) as described above.

The pellets of the present invention preferably exhibit a dissolution release profile, when measured as a plurality of pellets, wherein less than 25% of tamsulosin, preferably less than 15% of tamsulosin and most preferably less than 10% of tamsulosin is released during the first two hours in simulated gastric fluid in basket apparatus at 100 rpm. Accordingly, once the coated pellets of the present invention are ingested, tamsulosin is released into the body at a rate that is characterized by minimizing the release during the pellets' residence time in the stomach environment. More advantageously, the pellet core size and composition as well as the material and the relative amount of the coating are so selected that the resulting population of pellets exhibits at least one of the following release rates in simulated intestinal fluid (sometimes referred to herein as phosphate buffer of pH 6.8), using Ph.Eur.basket method at 100 rpm:
30-65%, preferably 40-60% of the tamsulosin in one hour, and/or more than 80% of the tamsulosin in six hours.

More preferably, the pellets satisfy all two release rates.

For clarity sake, the composition of simulated gastric fluid (SGF) and simulated intestinal fluid (SIF), although well known in the art as standard solutions, are set forth below:

SGF (USP Simulated Gastric Fluid without pepsin) composition:

| | |
|---|---|
| HCl | qs pH 1.2 |
| NaCl | 0.2% |
| water | qs 1000 ml |

SIF (USP Simulated Intestinal Fluid without pancreatin) composition:

| KH₂PO₄ | 6.8 | g |
|---|---|---|
| NaOH | qs pH 6.8 | |
| water | qs 1000 | ml |

The advantageous technique useful for making pellets of the present invention is extrusion-spheronization technique. In the preferred process, the calculated amount of the tamsulosine hydrochloride, which corresponds to 0.15-3.00 weight % of the total mass of the final pellet, is blended with the calculated amount of the pellet-forming carrier, e.g. with the microcrystalline cellulose, and the blend is mixed in a high-shear mixer with the aqueous solution or dispersion of the release-control agent, e.g. the acrylic polymer. The resulting wet granulate of tamsulosin in a carrier matrix is extruded and spheronized in the corresponding equipment with a corresponding sieve aperture, which is advantageously of about 1.0 mm. The formed wet pellet cores are then dried in a suitable drier, until the content of residual water is within the predetermined limit, which is advantageously between 2-10 mass %, preferably between 2-5 mass %.

The control of the residual water content in produced pellet cores may be made, for example, by taking samples of pellets and annealing them in an oven at 105° C., while measuring the weight loss.

The process of coating the pellet cores by the coating composition, which typically comprises an acid resistant acrylic polymer, may be performed in any suitable equipment such as a fluid bed coater, or a coating pan. The results of the coating procedure may be routinely checked by withdrawing a sample of the pellets and determining the release rate of tamsulosin in simulated gastric fluid as described above. However, if the desired amount of release is not achieved, the coating process of the remaining coated pellets, may be repeated until the desired result is obtained. It is indeed also possible to mix various sub-lots of coated pellets with different release rates to obtain a final lot exhibiting the desired rate. If one sub-lot does not yield the desired pellet size distribution, the negative effects can be made up with other sub-lots.

Once the coated pellets have been produced they are sieved through the sieve having the pore diameter of 1.4 mm. The fraction that passes through the sieve pores is collected, the fraction that does not pass is discarded. The entire population of the sieved pellets has then a size less than 1.4. mm. Optionally, such population may be further sieved through the sieve of the pore diameter of 0.3 mm and the fraction that passes the sieve and represents pellets with a size of less than 0.3 mm is discarded. Then the pellets in the final population have a size of less than 1.4 mm and at least 90% of them have a size of more than 0.3 mm as well.

The final population of pellets is stored in proper container for the use in making the final dosage forms.

As taught above, the produced pellets may be formulated into dosage units for co-administration of tamsulosin with other therapeutically active substances within a fixed dose combination. A suitable dosage unit is, for instance, an outer capsule, in which there is placed an inner capsule loaded by a composition comprising the drug, which is to be co-administered with tamsulosin, and a plurality of the above-defined tamsulosin-containing coated pellets comprising the entire tamsulosin dose are filled in the space between the inner and outer capsule. Accordingly, the unit dosage form may contain a plurality of pellets comprising the tamsulosin dose of between 0.1 to 1 mg of tamsulosin hydrochloride per unit, even more preferably 0.1, 0.2, 0.4 or 0.8 mg of tamsulosin hydrochloride per unit. The second drug is contained in the therapeutically effective dose as well.

The capsules of a suitable size may be made, e.g., from hard gelatin or hydroxypropyl methylcellulose. The size of the inner capsule is preferably so selected that the space between the surfaces of both capsules is at least 1.5 mm of width.

Such a unit dose is normally taken from 1 to 3 times daily, preferably once a day. In practice, the physician will determine the actual dosage and administration regimen, which will be the most suitable for the individual patient.

Capsules with coated pellets of the present invention comprising a unit dosage amount of tamsulosin may be delivered for immediate use in a suitable package comprising advantageously from 5 to 100 capsules. Such package may comprise a blister pack comprising advantageously 10, 14, 20, 28 or 30 capsules, or a plastic or glass container/bottle containing the same amounts of capsules. Any suitable pharmaceutically acceptable package material may be used in production the package unit.

Coated pellets for oral administration of tamsulosin according to the present invention may be used, for example, in the management of functional treatment of symptomatic benign prostatic hypertrophy or hyperplasia (BPH) or other disorders treatable by tamsulosin (the Disorders). The gastro-resistant coating and extended release of tamsulosin from pellet core assures that therapeutic concentration of tamsulosin in blood is maintained for sufficiently long time, without initial dumping in the stomach.

Accordingly, the present invention further provides a method for treating and/or preventing any one or more disorders which comprises orally administering an effective and/or prophylactic amount, to a sufferer in need thereof, of tamsulosin, which is formulated into a coated pellet as specified above. Preferably, the pellets of the invention are administered once a day, and more preferably after meal. Administration after food intake is advantageous because of better dispersion of pellets in the environment and minimizing damages of tissues of gastrointestinal tract.

The present invention also provides the use of the coated tamsulosin-comprising pellet as specified above, as well as the use of the above process for making the tamsulosin pellet composition itself, for the manufacture of a fixed-dose combination medicament for treating and/or preventing any one or more of the Disorders.

The invention is further illustrated by the following Examples, but should not be construed as being limited thereto.

### Example 1

### Tamsulosin Hydrochloride Enteric-Resistant Pellets with an Average Content 0.224% of Tamsulosin Hydrochloride

Pellet Composition:

| Ingredients | mg per capsule |
|---|---|
| pellet core: | |
| Tamsulosin•HCl | 0.400 |
| Eudragit L 30 D-55 | 8.250 |
| Triethylcitrate | 0.825 |
| Talc | 8.250 |
| Microcrystalline cellulose | 138.25 |
| Water (demineralized)¹ | 6.240¹ |
| Total mass of the pellet core | 162.215 |
| pellet coating | |
| Pellet cores | 162.215 |
| Eudragit L 30 D-55 | 10.815 |
| Talc | 4.325 |
| Triethylcitrate | 1.081 |
| Water (demineralized)² | —² |
| Total mass of the coating | 16.221 |
| Total mass of the pellets | 178.436 |

¹After drying to the content of volatiles of between 2-4%
²Fully removed during coating process

Manufacturing Process:
Tamsulosin hydrochloride was mixed in a high shear mixer with talc and microcrystalline cellulose to a form homogeneous powder blend
A suspension of Eudragit, triethyl citrate and water was prepared in a separate vessel
The suspension was added to the powder blend and the mixture was granulated at 95 rpm
The produced granulate was extruded and spheronised at the following setting:
Feeder speed 20 rpm
Impeller speed: 20 rpm
Sieve aperture: 1.0 mm
Shuttle box filing time: approx. 184 sec
Spheroniser speed: 500 rpm
Spheronizer time: 3 min
The formed pellets were dried in a fluid bed dryer until the loss on drying (LOD) value of between 2-4%
The coating suspension was prepared by mixing triethylcitrate, water, Eudragit L30 D-55 and talc
The pellets were placed in a fluid bed coater and coated at 60 C through a spray nozzle of 1.8 mm until the amount of the coating suspension corresponding to 50% of the mass of the core pellets was consumed (corresponds to 10% mass of the coating).
The coated pellets were sieved through a 1.4 mm screen

Pellet Coating Results:

The content of residual water, measured by moisture analyzer at 105 C is between 2 and 4%

The particle size distribution, measured by sieving using 1180, 850, 500 and 300 micrometer screens is: 98% of the particle size is between 300 and 1180 micrometers.

The dissolution profile in simulated gastric fluid: less than 10% in 2 hours.

The dissolution profile in pH 6.8 buffer (SIF): 40-60% in 1 hour, ≧80% in 6 hours.

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

1-18. (canceled)

19. A combination dosage form comprising a dose of tamsulosin physically separated from a dose of a testosterone-5α-reductase inhibitor, said combination dosage form comprising an inner capsule loaded with said dose of testosterone-5α-reductase inhibitor and an outer capsule surrounding the inner capsule and forming a space between the inner and outer capsules, wherein the space between the inner capsule and outer capsule is filled with said tamsulosin dose in the form of a population of tamsulosin pellets comprising tamsulosin hydrochloride uniformly dispersed in a carrier matrix, wherein said pellets:
(i) have a size of less than about 1.4 mm and, optionally at least 90% of the pellets have a size of larger than 0.30 mm;
(ii) an average content of tamsulosin hydrochloride in the population of pellets between about 0.15-3.00 weight percent, calculated on a dry pellet basis; and
(iii) have a dissolution release profile, when measured as a plurality of pellets, such that less than 25% of tamsulosin is released during the first two hours in simulated gastric fluid using Ph.Eur. basket method at 100 rpm, and 30-65% of the tamsulosin is released in one hour, or more than 80% of the tamsulosin is released in six hours, or both, in a phosphate buffer of pH 6.8, using Ph.Eur. basket method at 100 rpm.

20. The combination dosage form according to claim 19, wherein the testosterone-5α-reductase inhibitor is dutasteride or finasteride.

21. The combination dosage form according to claim 1, wherein said pellets comprise a core comprising tamsulosin hydrochloride uniformly dispersed in a carrier core matrix and a tamsulosin-free coating layer comprising an acid resistant acrylic polymer.

22. The combination dosage form according to claim 19, wherein the carrier matrix comprises:
a pellet forming carrier selected from microcrystalline cellulose, alpha lactose, dextrin, mannitol, or chitosan, alone or in combination, in an amount of 50-95 mass %, calculated on a dry pellet core basis;
a release control agent selected from water permeable acrylic polymers wherein the amount of the release control agent is preferably from 2.5 to 25 mass %, calculated on a dry pellet core basis; and
water in an amount from 2 to 10%, preferably 2 to 5%, calculated on a dry pellet core basis.

23. The combination dosage form according to claim 22, wherein said pellet core comprises 0.2-0.5% mass of tamsulosin hydrochloride, 50-95% mass of microcrystalline cellulose, 1-25% mass of the release control agent acrylic polymer(s), 2-10% mass of water, and 0-25% mass of other pharmaceutically acceptable excipients, calculated on a dry pellet core basis.

24. The combination dosage form according to claim 23, wherein said release control agent is selected from a copolymer of methacrylic acid, or an acrylic or methacrylic acid ester, or a combination of two or more.

25. The combination dosage form according to claims 21, wherein the acid-resistant polymer comprises an Eudragit L polymer.

26. The combination dosage form according to claim 21, wherein the composition of said outer layer coat comprises 25-95 mass % of said acid-resistant acrylic polymer, calculated on a dry basis.

27. The combination dosage form according to claim 21, wherein the mass of said outer layer coat, calculated on a dry pellet core basis, is within the range of 2.5-17 mass percent.

28. The combination dosage form according to claim 27, wherein said mass of said outer layer coat is within the range of 8-15 mass % of the weight of the dried pellet core.

29. The combination dosage form according to claim 19, wherein the dose of tamsulosin, calculated as tamsulosin hydrochloride, is within the range of 0.1 to 1 mg.

30. The combination dosage form according to claim 29, wherein said dose of tamsulosin is 0.1, 0.2, 0.4, or 0.8 mg.

31. The combination dosage according to claim 30, wherein the tamsulosin dose, calculated as tamsulosin hydrochloride, is 0.4 mg, and the average content of tamsulosin hydrochloride in the population of pellets is 0.224% weight percent, calculated on a dry pellet basis.

32. A method of treating benign prostatic hyperplasia, which comprises orally administering the combination dosage form according to claim 19 to a patient in need thereof.
